(19) United States
(12) Patent Application Publication
(10) Pub. No.: US 2022/0050099 A1
(43) Pub. Date: Feb. 17, 2022

(54) BIOCOMPATIBLE QUANTUM DOT SENSOR

(71) Applicants: Jonathan Veinot, St. Albert (CA); Christopher Jay T. Robidillo, Imus (PH)

(72) Inventors: Jonathan Veinot, St. Albert (CA); Christopher Jay T. Robidillo, Imus (PH)

(21) Appl. No.: 17/402,368

(22) Filed: Aug. 13, 2021

(30) Foreign Application Priority Data
Aug. 14, 2020 (CA) 3090115

(51) Int. Cl. G01N 33/543; C09K 11/02; C09K 11/06; C09K 11/59; G01N 21/64

(52) U.S. Cl. CPC G01N 33/54346; C09K 11/025; C09K 11/06; B82Y 20/00; G01N 33/54366; G01N 21/6428; C09K 11/59

(57) ABSTRACT

A sensor uses a combination of biocompatible quantum dots and an organic fluorophore in a controlled ratio. The organic fluorophore exhibits fluorescence of a first color, and the biocompatible quantum dots are sized to exhibit fluorescence of a second color different from the first color. The biocompatible quantum dots are functionalized with an organic coating arranged to chemically interact with a substance to quench the fluorescence of the quantum dots. The sensor exhibits a ratio of fluorescence of the quantum dots and the organic fluorophore from which a presence of the substance can be detected.

### TECHNICAL FIELD

Quantum dot sensors.

### BACKGROUND

Quantum dots may be used to produce sensors by employing pairs of quantum dots producing different respective colours in which the fluorescence of one of the pair of quantum dots is modified by the presence of the material that is to be sensed. In some known applications, pairs of CdTe or CdSe quantum dots may be used to detect TNT on a material. For instance, a pair of CdTe quantum dots, one emitting red fluorescence and the second emitting green fluorescence are used in a sensor with the red quantum dots embedded in silica nanoparticles. In the presence of TNT some of the green quantum dots may bind to the TNT, leading to quenching of the green fluorescence. The presence of the TNT is thereby detected by the relative reduction of the colour green in the fluorescence of the sensor.

Another application for quantum dot sensors is in detecting nerve agents. Nerve agents belong to a class of phosphorous-containing organic compounds broadly known as organophosphate esters (OPEs—also known as organic esters of phosphoric acids). These reagents are potent inhibitors of the neurologically important enzyme acetylcholinesterase (AChE) 1,2 by a mechanism that involves phosphorylation of the catalytically important serine residue in the enzyme active site. The associated diminished activity of AChE leads to a dramatic increase in levels of acetylcholine (ACh), a neurotransmitter that is released at nerve synapses and is important for normal nervous system function. The accumulation of toxic levels of ACh causes impaired cholinergic synapse transmission, leading to respiratory depression, prolonged seizures, and death. Nerve agents are toxic by all routes of exposure (e.g., inhalation, ingestion, contact with skin and eye) and are particularly potent percutaneous hazards. 1,2 Paroxon (PX), a p-nitrophenyl containing organophosphate, is one of the most potent organophosphate nerve agents. As such, it is now rarely used as a pesticide in the agriculture industry. Unfortunately, the possibility of human exposure cannot be ignored because PX has been weaponized. Parathion (PT), another extremely toxic p-nitrophenyl-containing organophosphate nerve agent, has been banned for use as pesticide in many jurisdictions (e.g., India, China, Japan, Thailand, New Zealand, Turkey, Sweden, United Kingdom, Russia). Despite its limited availability, numerous reports have implicated PT in poisoning and attempted suicides, and PT has also been employed in chemical warfare.

Infrastructure intensive methods for selective and sensitive determination of OPEs have been developed that include gas and liquid chromatography as well as mass spectrometry. Although accurate and sensitive, these methods suffer from limiting drawbacks such as the need for costly instrumentation and the necessity for highly trained technicians for operation these two factors alone preclude the convenient implementation of these methods. Recently, nano-material-based detection methods for OPEs have been developed.

These approaches rely on enzymesubstrate specificity for selective detection and signal amplification resulting from the nanostructures being enzyme carriers. Adding to the material function, the catalytic action of the enzyme on OPEs produces an electrochemical signal or photoluminescence (PL) quenching species, among others, that provides a mode of detection. In some instances, biosensor response is reliant upon the formation of an end product (e.g., hydrogen peroxide) that arises from a cascade of chemical reactions catalyzed by multiple enzymes. Though sensitive and selective, the reliance of these methods on the (combined) action of enzyme(s) adds complexity that could compromise biosensor performance (e.g., unwanted/unexpected denaturation and irreversible inactivation of enzymes). In addition, some reported fluorescent OPE sensors employ cytotoxic cadmium-based quantum dots (e.g., CdTe QDs) or involve the use of toxic heavy metal ions such as lead for detection. This heavy metal dependence potentially limits the utility of these systems in “real-world” sensing applications because their use can pose risks to human health.

### SUMMARY

In an embodiment there is a sensor for detecting a substance, the sensor comprising a combination of biocompatible quantum dots and an organic fluorophore in a controlled ratio, the organic fluorophore exhibiting fluorescence of a first colour, and the biocompatible quantum dots sized to exhibit fluorescence of a second colour different from the first colour, and the biocompatible quantum dots functionalized with an organic coating arranged to chemically interact with the substance to quench the fluorescence of the quantum dots.

In various embodiments, there may be included any one or more of the following features: the quantum dots comprising silicon nanoparticle quantum dots; the organic fluorophore comprising green fluorescent protein; the green fluorescent protein is mAmetrine 1.2; the poly(ethylene oxide) terminates in an alkoxide group where the pendant alkyl group is a C1, C2, C3, C4, C5, C6, C7, C8, C9, C10, C11, or C12 alkyl group, for example the poly(ethylene oxide) terminates in a methyl ether group; the organic coating comprises a water solubility enhancing component; the water solubility enhancing component comprises one or more of a carboxylic acid, primary amine, secondary amine, and alcohol; the organic coating comprises carboxylic acid; the organic coating comprises carboxylic acids including more than one type of alkyl group; and the organic coating may comprise carboxylic acid with a C12 alkyl group or comprise 10-undecenoic acid; the combination being provided in a solution; the combination is placed on a solid carrier; and the solid carrier comprises one of paper, polymer fiber membrane, glass fiber membrane, or polymer substrate.

In another embodiment there is a sensor for detecting a substance, the sensor comprising a combination of silicon nanoparticle quantum dots and an organic fluorophore in a controlled ratio, the organic fluorophore exhibiting fluorescence of a first colour, and the silicon nanoparticle quantum dots sized to exhibit fluorescence of a second colour different from the first colour, and the silicon nanoparticle quantum dots functionalized with an organic coating arranged to chemically interact with the substance to quench the fluorescence of the quantum dots.

There is in another embodiment a method of sensing a substance comprising providing a combination of biologically-compatible quantum dots and an organic fluorophore in a controlled ratio. The organic fluorophore exhibiting fluorescence of a first colour and the biologically-compatible quantum dots sized to exhibit fluorescence of a second colour different from the first colour. The biologically-compatible quantum dots functionalized with an organic coating arranged to chemically interact with the substance to quench the fluorescence of the biologically-compatible quantum dots. Applying a sample of a material to the combination of biologically-compatible quantum dots and the organic fluorophore. Exciting the combination of biologically-compatible quantum dots and the organic fluorophore. Detecting a produced fluorescence. Determining whether the produced fluorescence indicates the presence of the substance.

In another embodiment there is provided sensor for detecting a substance, the sensor comprising a combination of a biocompatible fluorescent nanoparticle that is responsive to the substance and an organic fluorophore in a controlled ratio, the organic fluorophore stable with respect to the substance, the organic fluorophore exhibiting fluorescence of a first colour, and the biocompatible fluorescent nanoparticle exhibiting fluorescence of a second colour different from the first colour, and the biocompatible fluorescent nanoparticle functionalized with an organic coating arranged to chemically interact with the substance to quench the fluorescence of the safe fluorescent nanoparticle.

These and other aspects of the device and method are set out in the claims.

### BRIEF DESCRIPTION OF THE FIGURES

Embodiments will now be described with reference to the figures, in which like reference characters denote like elements, by way of example, and in which:

FIG. 1A is a graphic illustrating the thermally induced hydrosilylation of 10-undecenoic acid and allyloxy poly(ethylene oxide) methyl ether with H—SiNPs.

FIG. 1B illustrate the FTIR spectrum.

FIG. 1C illustrate the carbonyl region FTIR spectrum.

FIGS. 1D-1F illustrate the Si 2p, C 1 s and O 1 s high-resolution X-ray photoelectron spectra of SiQDs, respectively.

FIG. 2A illustrates a DLS analysis size distribution analysis of water-soluble SiQDs.

FIG. 2B illustrates a silicon core size histogram of C₁₂H₂₅—SiQDs.

FIG. 2C illustrates a thermogravimetric profile of water-soluble SiQDs.

FIG. 3A illustrates absorbance (Abs), photoluminescence excitation (PLE), and photoluminescence emission (PL) spectra of SiQDs.

FIG. 3B illustrates absorbance (Abs), photoluminescence excitation (PLE), and photoluminescence emission (PL) spectra of mAm.

FIG. 3C illustrates absorbance (Abs), photoluminescence excitation (PLE), and photoluminescence emission (PL) spectra of a mixture consisting of SiQDs and mAm.

FIG. 4A illustrates PL spectra of SiQDs (1.1 μM) in the presence of increasing concentrations of mAm (λₑₓ=365 nm).

FIG. 4B illustrates PL spectra of mAm (1.8 μM) in the presence of increasing concentrations of SiQDs (λₑₓ=365 nm).

FIG. 4C-4H, respectively, illustrate the chemical structures of organophosphate nerve agents and p-Nitrophenol used in the experiments.

FIG. 5A illustrates PL spectra of SiQDs in the presence of increasing concentrations of PX (C_{SiQD}_{ₛ}=1.1 μM, λₑₓ=365 nm).

FIG. 5B illustrates PL spectra of SiQDs in the presence of increasing concentrations of PT (C_{SiQD}_{ₛ}=1.1 μM, λₑₓ=365 nm).

FIG. 5C illustrates PL spectra of mAm in the presence of increasing concentrations of PX (C_{mAm}=1.8 μM, λₑₓ=365 nm).

FIG. 5D illustrates PL spectra of mAm in the presence of increasing concentrations of PT (C_{mAm}=1.8 μM, λₑₓ=365 nm).

FIG. 6A shows Stern-Volmer plots (λₑₓ=365 nm) for PX, PT, and PN.

FIG. 6B shows plots of τ/τ° vs [Quencher] for PX, PT and PN.

FIG. 7A is a schematic diagram showing the operation of a sensor combining SiQDs and mAm.

FIGS. 7B-7D are photographs showing a series of solutions containing only SiQDs, only mAm, and SiQDs and mAm, respectively, in the presence of increasing micromolar concentrations of PX under UV illumination (λₑₓ=365 nm).

FIG. 8A illustrates PL spectra of solutions containing SiQDs and mAm in the presence of increasing concentrations of PX (C_{SiQD}_{ₛ}=1.1 μM, C_{mAm}=1.8 μM, λₑₓ=365 nm).

FIG. 8B shows a linear calibration plot for PX obtained by plotting I₅₂₅/I₆₃₅ against [Quencher] (C_{SiQD}_{ₛ}=1.1 μM, C_{mAm}=1.8 μM, λₑₓ=365 nm).

FIG. 8C illustrates PL spectra of solutions containing SiQDs and mAm in the presence of increasing concentrations of PX (C_{SiQD}_{ₛ}=1.1 μM, C_{mAm}=1.8 μM, λₑₓ=365 nm).

FIG. 8D shows a linear calibration plot for PT obtained by plotting I₅₂₅/I₆₃₅ against [Quencher] (C_{SiQD}_{ₛ}=1.1 μM, C_{mAm}=1.8 μM, λₑₓ=365 nm).

FIGS. 9A-9D illustrate plots of the effects of common interferents on the detection of (9A, 9B) PX and (9C, 9D) PT (C_{SiQD}_{ₛ}=1.1 μM, C_{mAm}=0.9 μM, λₑₓ=365 nm).

FIG. 10 shows PL spectra of solutions containing SiQDs and mAm in the presence of PX, PT, DZ, MT, CP, and PN (CSiQDs=1.1 μM, C_{mAm}=1.8 μM, λₑₓ=365 nm).

FIG. 11A shows unprocessed photographs of paper-based sensors spotted with PX, PT, DZ, MT, CP, PN, tap water, and mQ water, respectively under UV light illumination (λₑₓ=365 nm).

FIG. 11B illustrates green/red ratios obtained for each sample at a concentration of 100 μM.

FIG. 11C illustrates green/red ratios obtained for each sample at a concentration of 5 μM.

FIG. 12 is a flow chart showing an exemplary method of using a sensor.

### DETAILED DESCRIPTION

Immaterial modifications may be made to the embodiments described here without departing from what is covered by the claims.

There is a biocompatible fluorescent sensor in which two biologically compatible (biocompatible) fluorescing materials are combined, e.g. in a solution or in a solution applied to a solid substrate. One of the fluorescing materials is affected by the presence of one or more analytes, typically by quenching of its fluorescence, and may be referred to as a responsive fluorescent material. The fluorescence of the second material is substantially or wholly unaffected by the presence of the one or more analytes and may be referred to as a stable fluorescent material. A biocompatible substance is one that is not harmful to living tissue. An example of materials that are not biocompatible are toxic materials such as semiconductors formed from toxic metals. An example of a toxic metal that would not be a main component of a biocompatible sensor is Cadmium (Cd). Other materials that may not be biocompatible include heavy metals and potential or known carcinogens. Some materials not currently known to be toxic or carcinogens are suspected to be potentially unsafe. For example, it is uncertain whether germanium (Ge) quantum dots are safe. Additionally, some ions produced by metals, such as metallic quantum dots, may interfere with the fluorescence of one or both of the fluorescent materials in a sensor.

A combination providing two biologically compatible fluorescing materials may use a fluorescent protein as the materials which is unaffected by the presence of the analytes and a fluorescent nanoparticle based on a covalent network as the material which is affected by the presence of the analyte. Fluorescent proteins, of which many are known in the art, have the advantage of providing stable fluorescence which is unaffected by the presence of most materials. The barrel structure that is characteristic of these proteins can isolate the protein from chemical interactions that might affect the fluorescence. Fluorescent proteins also have the advantage that they are organic, safe and known to be biologically compatible. There is also a large spectral range of colours available for fluorescent proteins, so the fluorescence may be tuned for the application with reference to the second fluorescing material.

A fluorescent nanoparticle based on a covalent network may be used as a biocompatible fluorescing material which can be selected or prepared to have a suitably tuned fluorescent spectrum. Fluorescent nanoparticles based on a covalent network are more likely to be safe and biocompatible because: they can avoid the use of known toxic metals and heavy metals; the covalent bond networks are generally stable; and decomposition of the nanoparticles may produce benign or even beneficial products. For example, the decomposition of silicon nanoparticles may produce silicic acid, which is known to be safe for humans. Some potential fluorescent nanoparticles based on covalent networks may include silicon nanoparticle quantum dots, carbon nanoparticle quantum dots (C-dots), and encapsulated fluorescing dyes, such as fluorescent dyes encapsulated in core-shell silica nanoparticles. In the remainder of this specification, in some embodiments where a quantum dot material is described, the quantum dots might be substitutable with an encapsulated fluorescent dye.

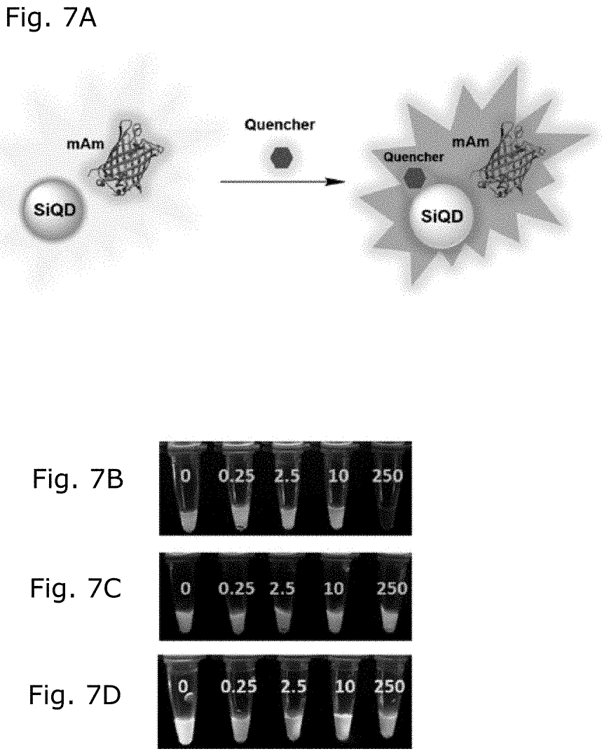

In an embodiment a quantum dot sensor may be used to detect a desired substance by utilizing two or more fluorescing materials in which at least one of the fluorescing materials comprises quantum dots. An exemplary illustration of a quantum dot sensor is shown in FIG. 7A. The exemplary sensor uses a biocompatible quantum dot, here a silicon quantum dot (SiQD), along with an organic fluorophore,here mAmetrine 1.2 (mAm). In this quantum dot sensor the fluorescence of the quantum dot is affected, directly or indirectly, by the presence of the material being sensed, for example by quenching of the quantum dot. A biocompatible quantum dot sensor can be produced from biocompatible fluorescing materials. Other possible biocompatible quantum dots may include carbon dots (C-dots) and ZnS quantum dots. Biocompatible quantum dots may include covalently bonded materials.

In this specification, “chemical interaction” between a first substance and second substance is interpreted to include direct or indirect interactions, including but not limited dipole-dipole interactions, hydrogen bonding and selective bonds. An indirect interaction might include an interaction mediated by one or more intermediate chemicals.

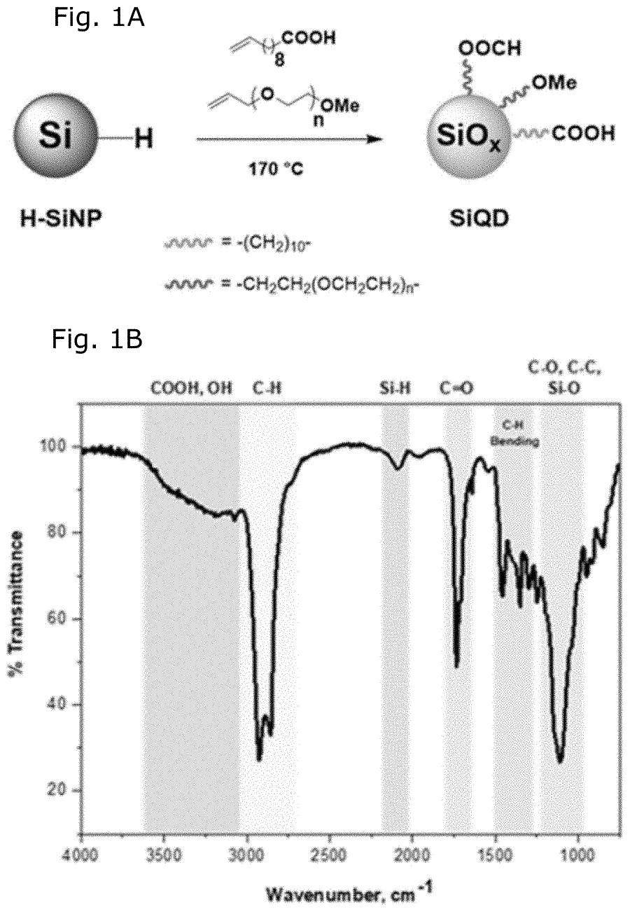

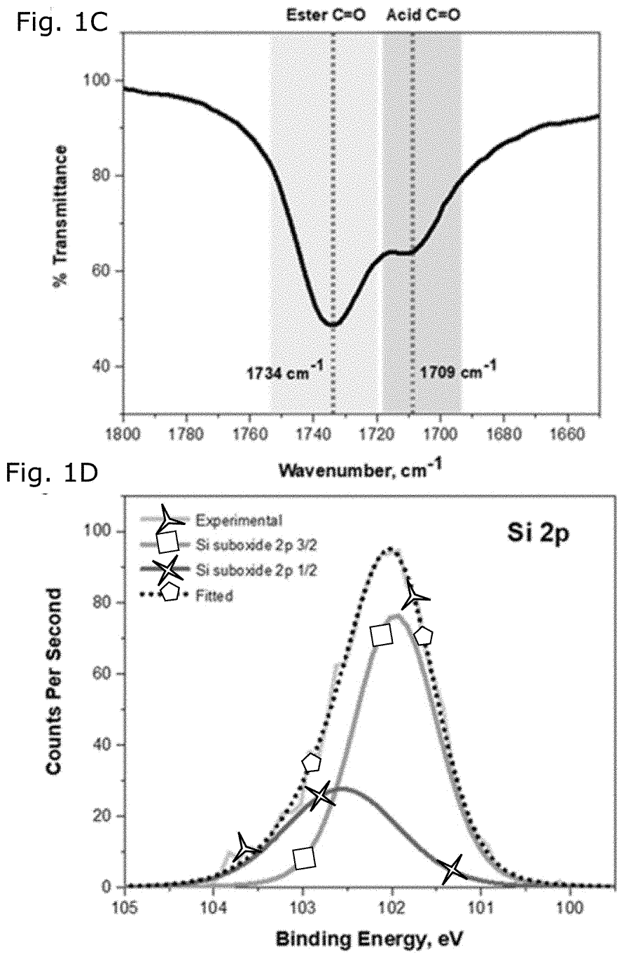

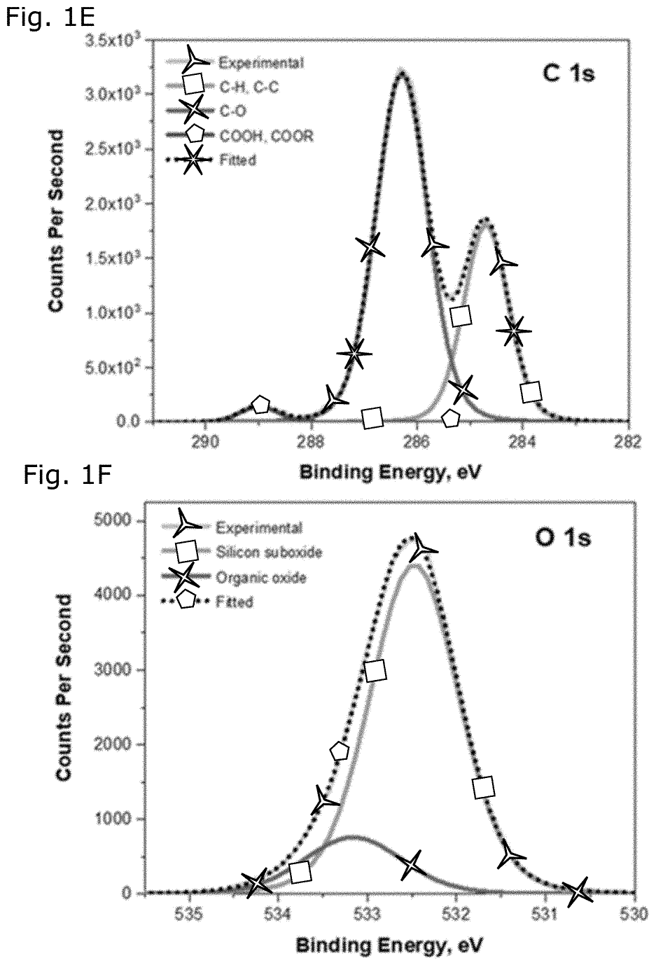

In a preferred embodiment of a biocompatible quantum dot sensor, a first fluorescing material comprises an organic fluorophore exhibiting fluorescence of a first colour, for example mAm as shown in the exemplary sensor shown in FIG. 7A and a second fluorescing material comprises silicon nanoparticle quantum dots (SiQD) as shown in FIG. 7A. The silicon nanoparticle quantum dots are sized to exhibit fluorescence of a second colour different from the first colour, and the quantum dots are functionalized with an organic coating (not shown in FIG. 7A) arranged to chemically interact with the desired substance to quench the fluorescence of the quantum dots. FIG. 1A shows an exemplary quantum dot formed of SiOₓ and having an organic coating including, in this example, —CH₂CH₂(OCH₂CH₂)ₙ— chains terminated by OOCH or OMe groups, and —(CH₂)₁₀— chains terminated by COOH.

In some embodiments, the photoluminescence of silicon nanoparticle quantum dots may be used to detect nerve agents such as PX and PT. A silicon quantum dot may be functionalized with an organic coating arranged to chemically interact with to the nerve agent to quench the fluorescence of the silicon nanoparticle quantum dots. The photoluminescence of silicon nanoparticle quantum dots might similarly be used to detect other materials by functionalizing the silicon quantum dots with an organic coating arranged to chemically interact with those other materials. The chemical interaction resulting in quenching of the fluorescence may comprise dipole-dipole interactions or bonding between the nerve agent and the organic coating.

In some such embodiments, the organic coating may comprise poly(ethylene oxide). Poly(ethylene oxide is also known as polyethylene glycol (PEG). The poly(ethylene oxide) may be terminated by an alkoxide group where the pendant alkyl group is a C1 group and may comprise methyl. In experiments completed to test the silicon quantum dot sensor, a methyl ether group was selected. The poly(ethylene oxide) may also terminate in an alkoxide group where the pendant alkyl group is appropriately selected from a C1, C2, C3, C4, C5, C6, C7, C8, C9, C10, C11, or C12 alkyl group.

In some such embodiments, the organic coating may comprise a coating component for rendering the particles water soluble. In experiments completed to test the silicon quantum dot sensor, a carboxylic acid was selected for the organic coating to make the particles water soluble. Carboxylic acids are not always effective to make particles water soluble, but were found to be effective in this case when combined with the poly(ethylene oxide). Other components that could be used for this purpose include a primary amine, secondary amine or alcohol. The carboxylic acids may include more than one type of alkyl group, e.g. a C3, C4, C5 . . . C12. C12 is believed to provide the best performance. However, in a tested embodiment, 10-undecenoic acid was used.

The combination of silicon quantum dots and an organic fluorophore may be prepared in a solution and the solution may be applied to a solid carrier. A variety of solid carriers may be utilized. A suitable solid carrier may include paper, such as standard filter paper. Other suitable solid carriers may include polymer fiber membrane, glass fiber membrane, or polymer substrates, among others.

Analytes that may be detected by sensors according to these embodiments may include substances containing nitroaromatic groups. In a preferred embodiment, the analytes that are detected by the sensor include organophosphate esters including the nerve agents PX and PT. In some embodiments, a sensor may be produced to detect other substances containing nitroaromatic groups such as TNT.

The sensor may comprise a paper impregnated with a biocompatible fluorescent sensor, such as a solution of non-toxic silicon-based quantum dots and green fluorescent protein for detecting nitro-containing nerve agents. Signal output may be generated, for example, through the use of a smartphone camera and evaluated using a smartphone app. The ratiometric platform offers the benefit of visual detection and probe concentration-independent response. In some embodiments, the sensor offers a quick and cost-effective means for inspecting items, objects or samples that may be contaminated with nerve agents. For example, it can be used for detecting nerve agent insecticides on bult produce like vegetables and fruit. The sensor may be capable of detecting explosives such as TNT as well. As such, it can also be employed by law enforcement, border control, and airport security personnel in assessing suspicious packages and luggage—those which potentially contain nerve agents and explosives—en masse in airports and other ports of entry. Although a smartphone and smartphone app can be used to detect contaminants such as nerve agents and explosives, various other apparatus can be used to detect the relevant items, including any piece of technology that includes both a camera and a processor capable of detecting the signal output. The processor and camera may be specifically designed for a specific application, such as at an airport security check.

### Experimental Set Up

Chemicals. A methyl isobutyl ketone solution of hydrogen silsesquioxane (HSQ, trade name Fox™-17, Dow Corning™) was evaporated to dryness to yield a white solid. Aqueous electronics grade hydrofluoric acid (49%) was used as received from J. T. Baker™. Allyloxy poly(ethylene oxide) methyl ether (9-12 ethylene oxide units, MW ˜450 g mol⁻¹, ρ=1.076 g mL⁻¹, Gelest™), 10-undecenoic acid (MW=184.28 g mol⁻¹, ρ=0.912 g mL⁻¹), paraoxon (PX), parathion (PT), diazinon (DZ), malathion (MT), chlorpyrifos (CP), p-nitrophenol (PN), 4-(2-hydroxyethyl)-1-piperazineethanesulfonic acid (HEPES) (Millipore Sigma™) were used as received. All other reagents and solvents used were of analytical grade unless otherwise specified.

Preparation of Oxide-Embedded Silicon Nanoparticles. A composite of oxide-embedded silicon nanoparticles (SiNPs) was prepared following a known method. HSQ (1 gram) was thermally processed upon heating in a standard tube furnace to 1100° C. for 1 h in a 5% H2:95% Ar atmosphere. This procedure yielded silicon oxide-embedded inclusions of elemental silicon with dimensions of circa 3 nm. This composite was further annealed for 1 h at 1200° C. in an Ar atmosphere to grow the inclusion dimensions to circa 6 nm. The resulting “6 nm composite” was processed into finely powdered stock material, as described previously.

Synthesis of Hydride-Terminated Silicon Nanoparticles (HSiNPs). Hydride-terminated silicon nanoparticles (H—SiNPs) were liberated from their oxide matrix by ethanolic HF etching. Synthesis of Silicon-Based Quantum Dots (SiQDs). Mixed surface acid-terminated poly(ethylene oxide)-coated silicon-based quantum dots (SiQDs) were prepared through thermally induced hydrosilylation.

Protein Expression and Purification of mAmetrine 1.2 (mAm). DNA encoding mAmetrine 1.2 in pBAD/His B vector (Thermo Fisher Scientific™) was transformed into electrocompetent Escherichia coli strain DH10B (Invitrogen). mAmetrine 1.2 is described in greater detail in Ding, Y.; Ai, H.; Hoi, H.; Campbell, R. E. Föster Resonance Energy Transfer-Based Biosensors for Multiparameter Ratiometric Imaging of Ca2+ Dynamics and Caspase-3 Activity in Single Cells. Anal. Chem. 2011, 83, 9687-9693. Transformed E. coli were then cultured on Lennox Broth (LB) agar plates supplemented with 400 μg/mL of ampicillin (Thermo Fisher™) and 0.02% L-arabinose (Alfa Aesar) at 37° C. overnight. Single colonies from the transformed bacteria were used to inoculate 200-500 mL of LB supplemented with 100 μg/mL of ampicillin and 0.02% L-arabinose and cultured at 37° C. for 24 h. After culturing, bacteria were harvested by centrifugation at 8000 rpm for 10 min and resuspended in lysis buffer (50 mM Tris-HCl, 100 mM NaCl, 5% glycerol, 1 mM imidazole, pH 8.0). Cells were lysed using sonication and then clarified by centrifugation at 14 000 rpm for 30 min. The cleared lysate was incubated with Ni—NTA beads (G-Biosciences™) on a rotary platform for at least 1 h. The lysatebead mixture was then transferred to a polypropylene centrifuge column and washed with 5-packed column volumes of wash buffer (lysis buffer with 20 mM imidazole, pH 8.0) before elution using Ni—NTA elution buffer (lysis buffer with 250 mM imidazole, pH 8.0). Purified mAm was concentrated and buffer-exchanged into 20 mM HEPES (pH 7.0) using 10 kDa centrifugal filter units (Millipore™). All steps were carried out at 4° C. or on ice. Protein concentration was measured by A280 using an extinction coefficient of 31 000 M⁻¹ cm⁻¹.

Characterization of SiQDs. The SiQDs were characterized using Fourier transform infrared spectroscopy (FTIR), X-ray photoelectron spectroscopy (XPS), bright-field transmission electron microscopy (TEM), dynamic light scattering (DLS) analysis, and absorption spectroscopy, as described elsewhere. Thermogravimetric analysis (TGA) was performed from 25 to 800° C. using a TGA/DSC 1 STARe System (Mettler Toledo™) at a heating rate of 10° C. min⁻¹ under Ar flow.

Photoluminescence excitation (PLE) and emission (PL) spectra of the samples were recorded using a SpectraMax i3x multimode microplate reader. Time-resolved PL spectroscopy was performed. The decay data were modeled using the stretched exponential function I=A exp[(−(t/τ)β]+dc, where the fitting parameters are A, τ, β, and the dc offset. For this intensity decay function, the mean time constant is given by <τ>=τ·Γ[2/β]/Γ[1/β] and the mean decay time is <t>=τ·Γ[3/β]/Γ[2/β]. The fits were performed in MATLAB™ using the trust region algorithm with unweighted minimization of the sum of the squares of the residuals. PL quantum yield measurements were performed, as described previously.

Effect of mAm on the PL of SiQDs. Solutions (Vtotal=100 μL) containing 1.1 μM SiQDs and increasing concentrations (0, 0.5, 0.9, 1.8, 3.7 μM) of mAm were prepared by dilution of stock SiQD and mAm solutions with 20 mM HEPES buffer (pH 7.0). The PL spectra of the solutions were then measured at an excitation wavelength of 365 nm. Experiments were performed in triplicates.

Effect of SiQDs the PL of mAm. Solutions (Vtotal=100 μL) containing 1.8 μM mAm and increasing concentrations (0, 0.3, 0.6, 1.1, 2.2 μM) of SiQDs were prepared by dilution of stock SiQD and mAm solutions with 20 mM HEPES buffer (pH 7.0). The PL spectra of the solutions were then measured at an excitation wavelength of 365 nm. Experiments were performed in triplicates.

Effect of Quenchers on SiQD Photoluminescence. Solutions (Vtotal=100 μL) containing 1.1 μM SiQDs and increasing concentrations (0, 2.5, 5, 10, 20, 40 μM) of quencher in question (PX, PT, and PN) were prepared by dilution of 50 μM quencher solution with 20 mM HEPES buffer (pH 7.0). The PL spectra of the solutions were then acquired upon excitation at 365 nm. Stern-Volmer plots were constructed by plotting the ratio of PL intensities in the absence and presence of quencher at 635 nm, I°/I, against the concentration of quencher. Experiments were performed in triplicates. The ratio of PL lifetimes in the presence and absence of quencher, τ/τ°, was also plotted against the concentration of the quencher.

Effect of PX and PT on mAm Fluorescence. Solutions (Vtotal=150 μL) containing 1.8 μM mAm and increasing concentrations (0, 5, 25, 100 μM) of quencher (PX, PT) were prepared by dilution of 50 or 200 μM quencher solution with 20 mM HEPES buffer (pH 7.0). The PL spectra of the solutions were then measured at an excitation wavelength of 365 nm. Experiments were performed in triplicates.

Effect of PX and PT on the Photoluminescence of Mixtures of SiQDs and mAm. Solutions (Vtotal=150 μL) containing 1.1 μM SiQDs, 1.8 μM mAm, and increasing concentrations (i.e., 0, 2.5, 5.0, 10.0, 15.0, 20.0, 30.0, 40.0, 50.0, 75.0, 100.0 μM for PX; 0, 0.01, 0.1, 0.25, 0.5, 1.0, 2.5, 5.0, 10.0, 15.0, 20.0, 30.0, 40.0 μM for PT) of quencher were prepared by dilution of 5, 50 or 200 μM quencher solution with 20 mM HEPES buffer (pH 7.0). The PL spectra of the solutions were measured at an excitation wavelength of 365 nm. The ratios of the PL intensity at 525 to the intensity at 635 nm, I₅₂₅/I₆₃₅, were plotted against the concentration of the quencher to obtain a straight line. This plot was then used for the quantification of PX and PT. Experiments were performed in triplicates.

Analysis of Solutions with Known PX and PT Concentrations. Solutions (Vtotal=150 μL) containing 1.1 μM SiQDs, 1.8 μM mAm, and 25.0 μM PX (or PT) were prepared upon dilution of 50 μM quencher solution with 20 mM HEPES buffer (pH 7.0). The PL intensities of the solutions at 525 and 635 nm were then measured and the ratio I₅₂₅/I₆₃₅ evaluated and used to determine the concentration of PX or PT from the linear calibration plots obtained above. Experiments were performed in triplicates.

Effect of Interferents on the Detection of PX and PT Using SiQDs and mAm. Solutions (Vtotal=115 μL) containing 1.1 μM SiQDs and 0.9 μM mAm and 0.30 mM of the interferents (Salts: KCl, KNO₂, KNO₃, K₂CO₃, K₃PO₄, Na₂SO₄, NaCl, NaF, NH₄Cl, MgCl₂, Ca(NO₃)₂; Organics: alanine (Ala), lysine (Lys), arginine (Arg), malonic acid (MA), sodium acetate (Acet), sodium citrate (Cit), glucose (Glc), sucrose (Suc)) were prepared by dilution of corresponding 1.0 mM solution with 20 mM HEPES buffer (pH 7.0). The PL intensities of the solutions were then measured at an excitation wavelength of 365 nm and the ratio I₅₂₅/I₆₃₅ evaluated. Afterward, 35 μL of 100 μM quencher solution was added to each solution, the PL intensities measured, and the ratio I₅₂₅/I₆₃₅ after the addition of the quencher evaluated. The value of the ratio R=(I₅₂₅/I₆₃₅) after/(I₅₂₅/I₆₃₅) before for each solution was then used to determine the effect of interfering ions on the analysis by comparing it to values obtained for positive (115 μL solution containing 1.1 μM SiQDs and 0.9 μM mAm in HEPES buffer+35 μL 100 μM quencher solution) and negative (115 μL solution containing 1.1 μM SiQDs and 0.9 μM mAm in HEPES buffer+35 μL mQ water) controls. Experiments were performed in triplicates.

Selectivity of the Detection Method for PX and PT. Solutions (Vtotal=150 μL) containing 1.1 μM SiQDs, 1.8 μM mAm, and 20 μM organophosphate (PX, PT, DZ, MT, CP) or 20 μM PN were prepared by dilution of corresponding 100 μM organophosphate or PN solution with 20 mM HEPES buffer (pH 7.0). The PL spectra of the solutions were then measured at an excitation wavelength of 365 nm. Experiments were performed in triplicates.

Detection of PX and PT Using Paper-Based Sensors Containing SiQDs and mAm. Paper-based sensors were prepared by dipping filter paper pieces (1.0×0.4 cm2) in a solution containing 58 μM SiQDs and 36 μM mAm. After drying the papers in air for 30 min, 1 μL of organophosphate solution (5 and 100 μM; PX, PT, DZ,MT, CP), 1 μL of PN solution (5 and 100 μM), 1 μL of Edmonton municipal tap water, and 1 μL of mQ water were spotted on separate papers (Note: Tap water was chosen because it best approximates the real-world solution vehicle of the target analyte). After 1 min, the papers were exposed to a UV flashlight (λ=365 nm) that was held 15 cm from the paper surface, and photographs of the luminescence were acquired using a Canon Powershot™ SX730 HS camera with an ISO value of 3200 (exposure time= 1/200− 1/500 s). The Color Picker™ app from Ratonera Inc. downloaded on an Android smartphone from the Google Play™ Store was used to analyze the image into its component red/green/blue values. Green values were normalized to corresponding red values to obtain green/red ratios. Five points at the center of each image were evaluated to provide a statistical average corresponding to one experiment. Experiments were performed in five replicates.

### Results and Discussion

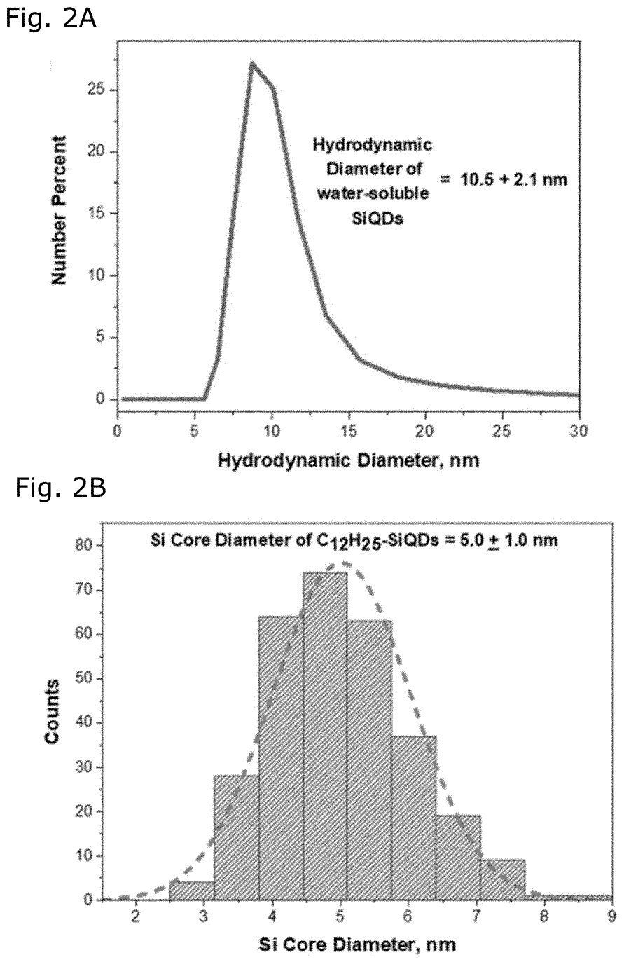

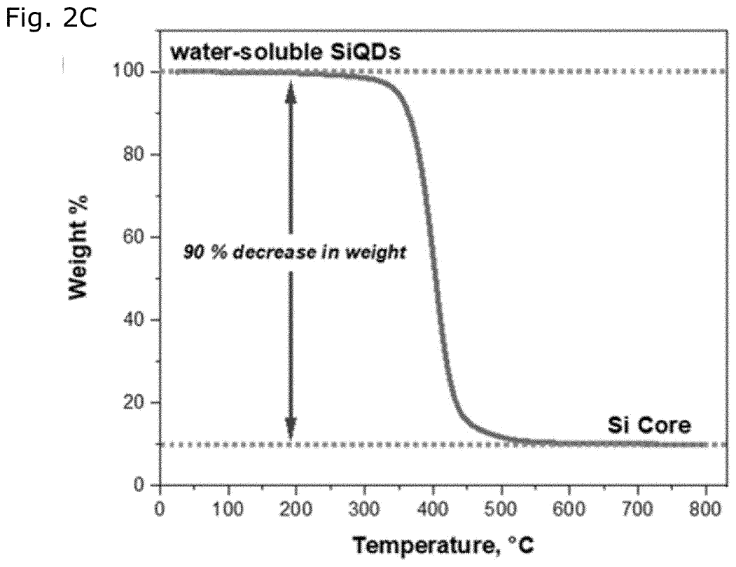

FIG. 1A summarizes the preparation of water-soluble Si-based quantum dots (SiQDs). Briefly, 10-undecenoic acid and allyloxy poly(ethylene oxide) methyl ether were linked to the surfaces of 6 nm hydride-terminated silicon nanoparticles (SiNPs) via thermally induced hydrosilylation at 170° C. The SiNPs were coated with poly(ethylene oxide) to render them water soluble and resistant to nonspecific protein adsorption. Successful surface functionalization was confirmed using Fourier transform infrared (FTIR) spectroscopy that shows the presence of aliphatic sp³ C—H stretching peak at 3000-2800 cm⁻¹ and characteristic carboxylic acid hydroxyl and carbonyl features centered at circa 3000 and 1709 cm⁻¹, respectively (FIGS. 1B and 1C). The diminished intensity of the peak at circa 2100 cm⁻¹ routinely assigned to Si—Hx stretching and the intense and broad peak at approximately 1100 cm⁻¹ characteristic of C—O and C—C stretches are also consistent with successful covalent functionalization. An additional carbonyl-based feature is noted at circa 1734 cm⁻¹ that arises from formate ester groups resulting from thermal oxidation of the poly-(ethylene oxide) moieties. Poly(ethylene oxide) is believed to react with trace oxygen at elevated temperatures forming hydroperoxides that then undergo β-scission into formate esters and hemiacetals. Characteristic bending —CHₓ— vibrations resulting from both immobilized undecanoic acid and poly(ethylene oxide) moieties are also noted in the 1500-1300 cm⁻¹ region of the FTIR spectrum. X-ray photoelectron spectroscopy (XPS) revealed that the SiQDs used in this study are made up of silicon suboxides as evidenced by the Si 2p_{3/2} peak at circa 101.8 eV (FIG. 1D). It is reasonable that these suboxides result from exposure of the silicon core to water during aqueous workup (e.g., extraction, dialysis, centrifugal filtration) and subsequent storage. The O 1 s emission at circa 532.3 eV corresponding to silicon-bonded oxygen atoms also supports the presence of silicon suboxides (FIG. 1F). Consistent with FTIR analyses, successful surface functionalization is further confirmed by the presence of C 1 s peaks centered at circa 284.8, 286.3, and 289.0 eV (FIG. 1E) that correspond to aliphatic C—C/C—H, aliphatic C—O, and carboxyl groups, respectively. We also note an O 1 s associated emission at circa 533.2 eV that corresponds to the O atoms of poly(ethylene oxide) (FIG. 1F). Aqueous phase dynamic light scattering (DLS) analysis of the SiQDs revealed an average hydrodynamic diameter of 10.5±2.1 nm (FIG. 2A). This value is consistent with the core of the SiQDs being coated with hydrophilic poly(ethylene oxide) moieties and surrounded by a water solvation sphere. Imaging the present water-soluble SiQDs using bright-field transmission electron microscopy (TEM) was unsuccessful because particles were highly agglomerated—this is again consistent with the SiQDs being functionalized with poly-(ethylene oxide). We also note a substantial and significant amount of organic content (i.e., 90%) in the present SiQDs as indicated by the thermogravimetric profile (FIG. 2C) that presumably arises from surface polymer functionalization and would be expected to preclude accurate determination of the particle core dimensions using electron microscopy. To better interrogate the dimensions of the particle core, we chose to prepare dodecyl-terminated SiQDs (C₁₂H₂₅—SiQDs) using HSiNPs obtained from the identical composite batch and etching conditions used to prepare the present water-soluble SiQDs. The C₁₂H₂₅—SiQDs were then analyzed with brightfield TEM, and their mean diameter (i.e., 5.0±1.0 nm; FIG. 2B) was assumed to provide a good approximation of the core dimensions of the water-soluble SiQDs. This value provided an estimated molar mass of the silicon core, which when combined with the thermogravimetric analysis data, gave access to the solution concentration of the water-soluble SiQDs.

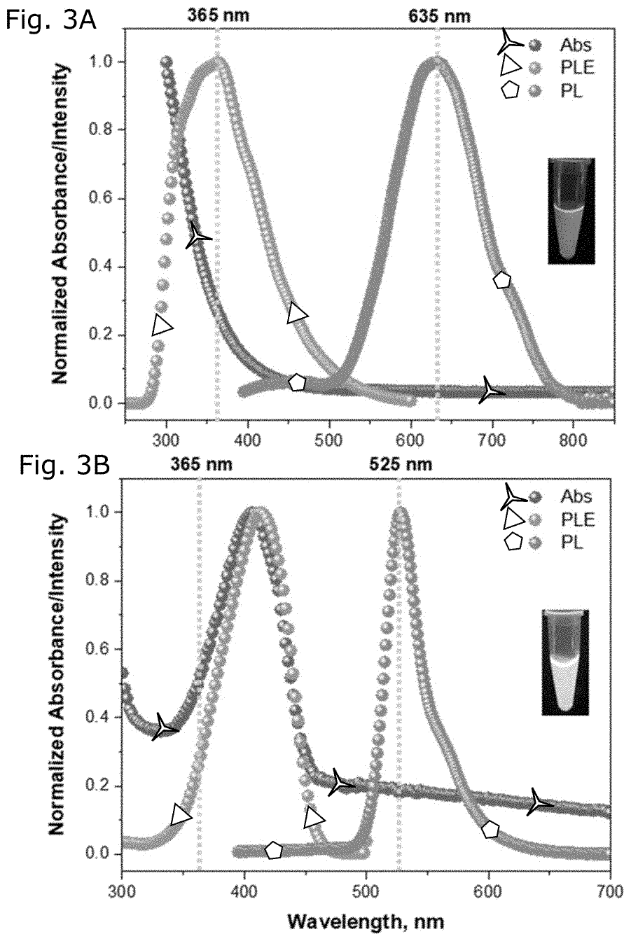

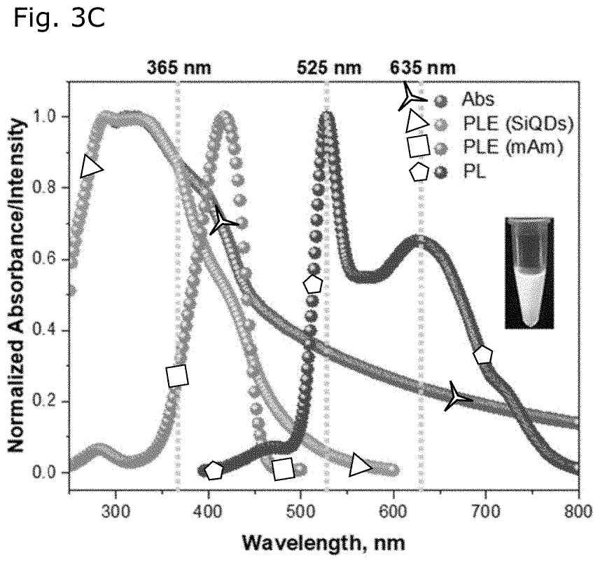

FIG. 3A-3C show the optical spectra of SiQDs, mAmetrine 1.2 (mAm), and a mixture of SiQDs and mAm. The SiQDs exhibit strong absorption at wavelengths shorter than 400 nm, have a PLE maximum at circa 365 nm, and a PL maximum at circa 635 nm. They also have a PL quantum yield of 9.7% and a long-lived excited-state lifetime of 58.4 μs; these observations are consistent with an indirect band gap silicon-based emitter. The inset in FIG. 3A demonstrates that an aqueous solution of SiQDs exhibits visibly detectable orange PL. In contrast, the fluorescent protein employed in this study, mAm, shows absorbance and PLE maxima at circa 410 nm and a green PL maximum at circa 525 nm (FIG. 3B, inset). A mixture of SiQDs and mAm appears yellow upon visible inspection and exhibits PL maxima at 635 and 525 nm (i.e., corresponding to each individual emitter and consistent with negligible interaction).

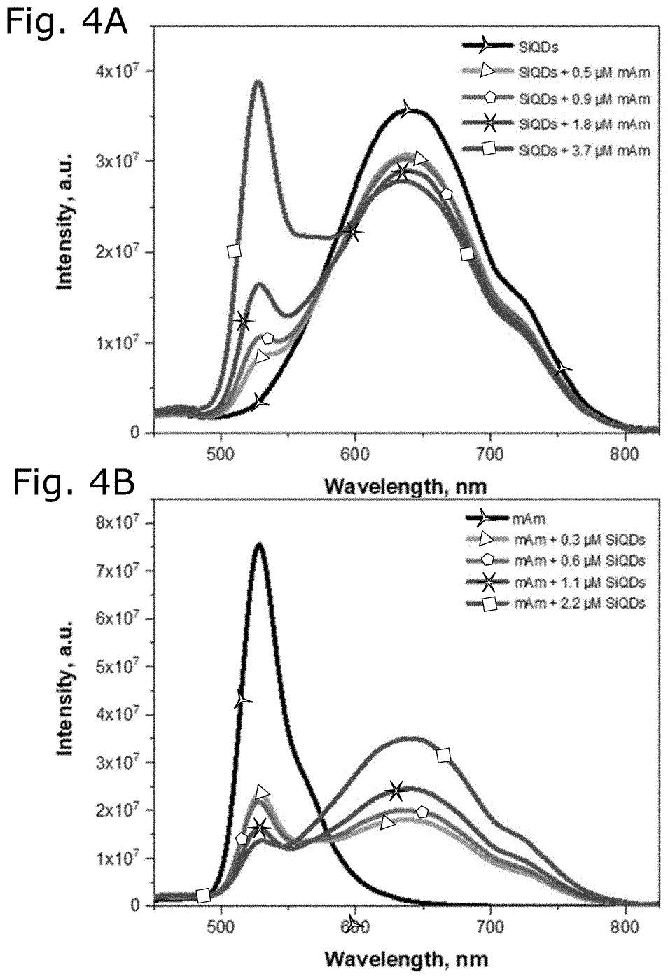

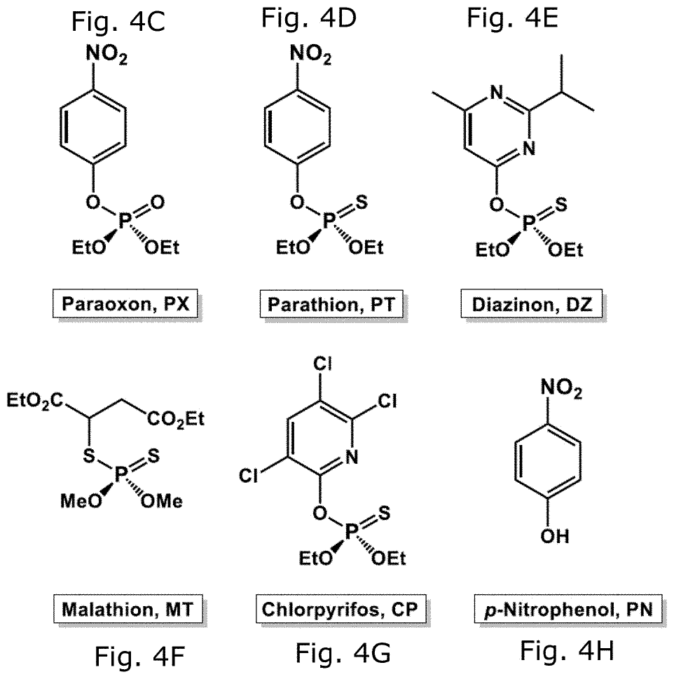

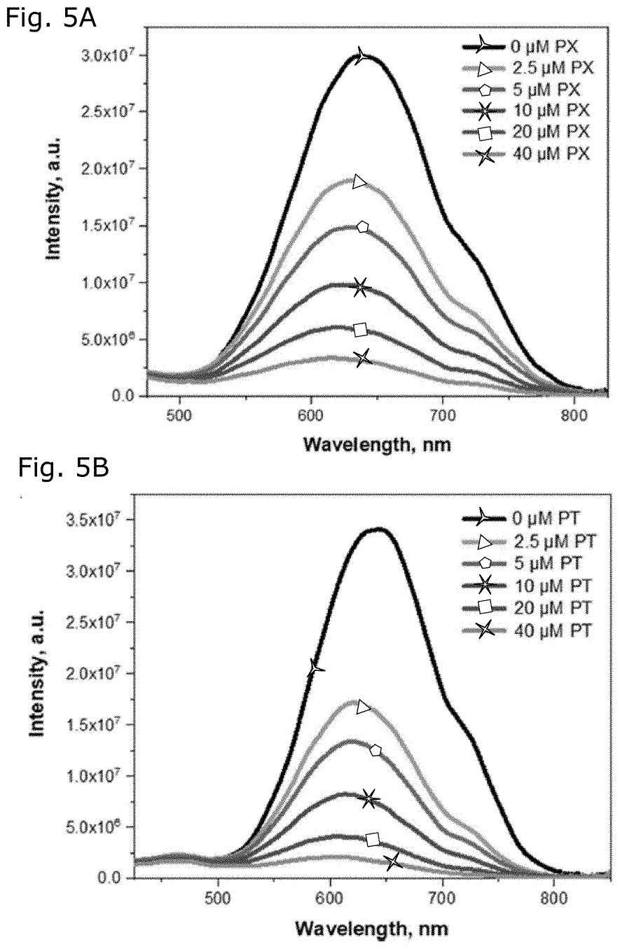

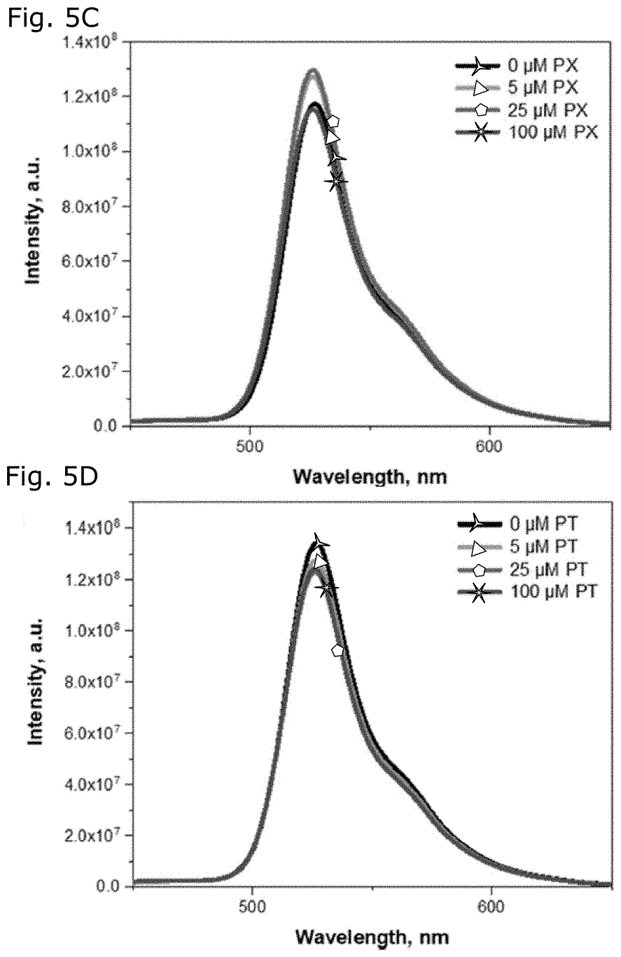

We subsequently investigated the behavior of each emitter in the presence of the other through rational concentration variation of one while maintaining the concentration of the other constant (FIGS. 4A and 4B). FIG. 4A shows the PL intensity of the SiQDs decreased with increasing mAm concentration. Similarly, the mAm PL intensity decreased in the presence of increasing SiQD concentration. This phenomenon has been observed for fluorophore mixtures and is reasonably attributed to the overlap of the PLE spectra of the two emitters and a resulting “competition” for incident excitation photons (i.e., an inner-filter effect). Photoluminescent sensors offering detection of high-energy nitro-based explosives based upon SiQDs as well as porous silicon particles have been reported. In this study, we extend the SiQD sensing repertoire by taking advantage of the tendency of nitroaromatic organic compounds to quench their PL and fabricated a ratiometric sensor based upon SiQD and mAm emitters for p-nitrophenyl-containing organophosphate nerve agents paraoxon (PX) and parathion (PT) (FIGS. 4C and 4D). To do so effectively, it was first necessary to determine the effect of PX and PT on the PL response of each emitter independently. Subsequently, we determined the effect of the addition of PX and PT on the PL of SiQD/mAm mixtures. FIGS. 5A and 5B show that PX and PT quench SiQD PL, whereas FIGS. 5C and 5D show that mAm PL is comparatively unaffected. In this document, the word “ratiometric” means that the output of the sensor is based on the ratio of different types of fluorescence.

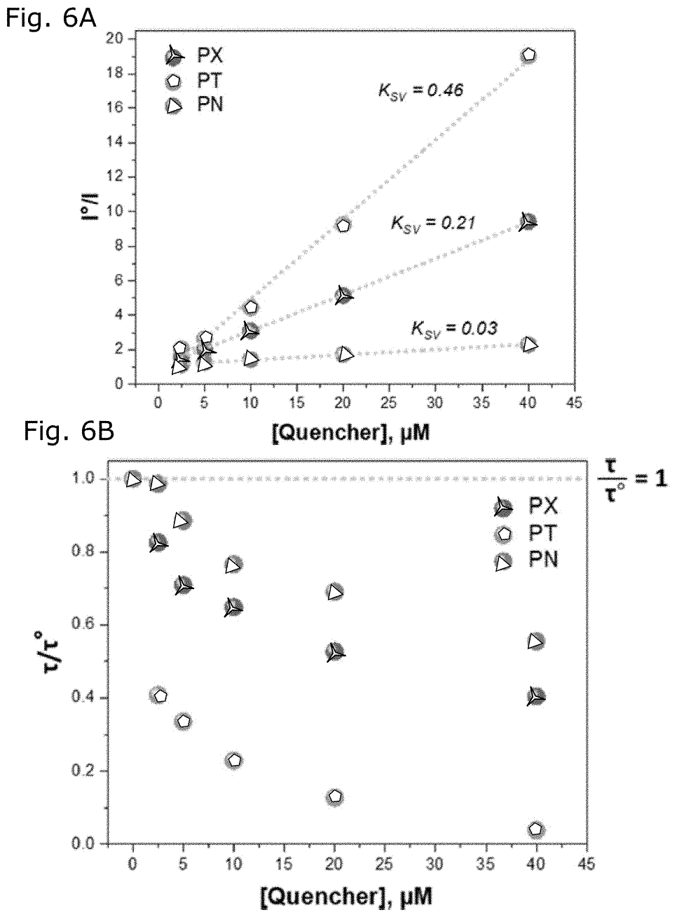

To better understand the response of the present sensing motif, we determined the mechanism by which PX, PT, and p-nitrophenol (PN) quench the SiQD PL through steady-state and time-resolved PL measurements. Corresponding Stern-Volmer plots (I°/I vs [Quencher]) for PX, PT, and PN shown in FIG. 6A yielded linear relationships with Stern-Volmer constants (KSV=slope) of 0.21, 0.46, and 0.03, respectively. These values suggest that PT is a more effective quencher than PX, which, in turn, is a much more effective quencher than PN. Plots of τ/τ° vs [Quencher] indicate that the excited-state lifetimes of the SiQDs decrease with increasing quencher concentration (FIG. 6B). The diminished SiQD lifetimes are consistent with PX, PT, and PN acting as dynamic quenchers that provide alternative relaxation pathways for the SiQDs—based upon past reports, it is reasonable that they are acting as electron acceptors.

FIG. 7A summarizes the general approach to sensing of p-nitrophenyl-containing organophosphate nerve agents through the selective quenching of SiQD PL. FIG. 7B shows the influence of increasing PX concentration on SiQD PL alone—no visually detectable change in PL intensity is noted until the PX concentration reaches 250 μM. Also, FIG. 7C shows that PX does not quench the PL of mAm. In contrast (FIG. 7D), when a mixture of SiQDs and mAm is exposed to varied PX concentrations, the changes in optical response are striking. The PL arising from a solution containing SiQDs and mAm clearly changes from yellow to green with increasing concentration of PX.

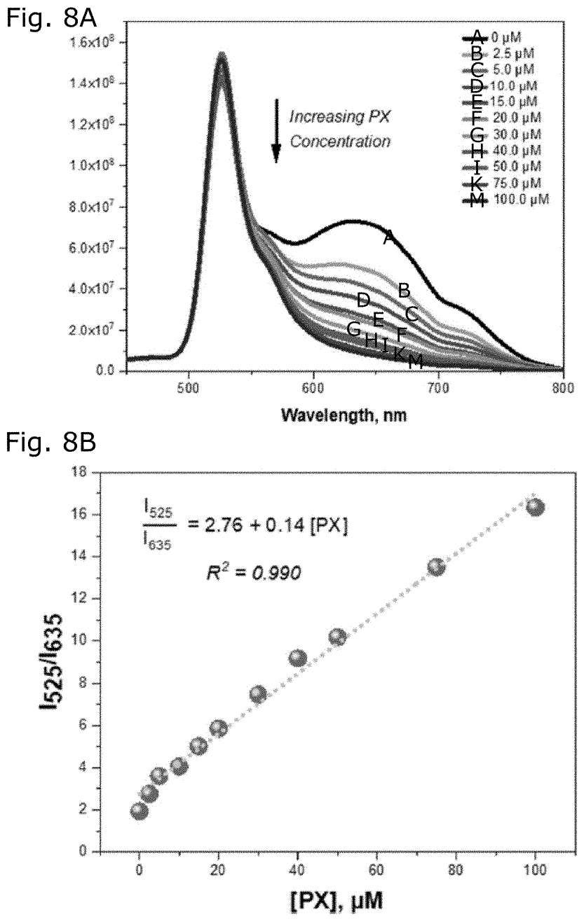

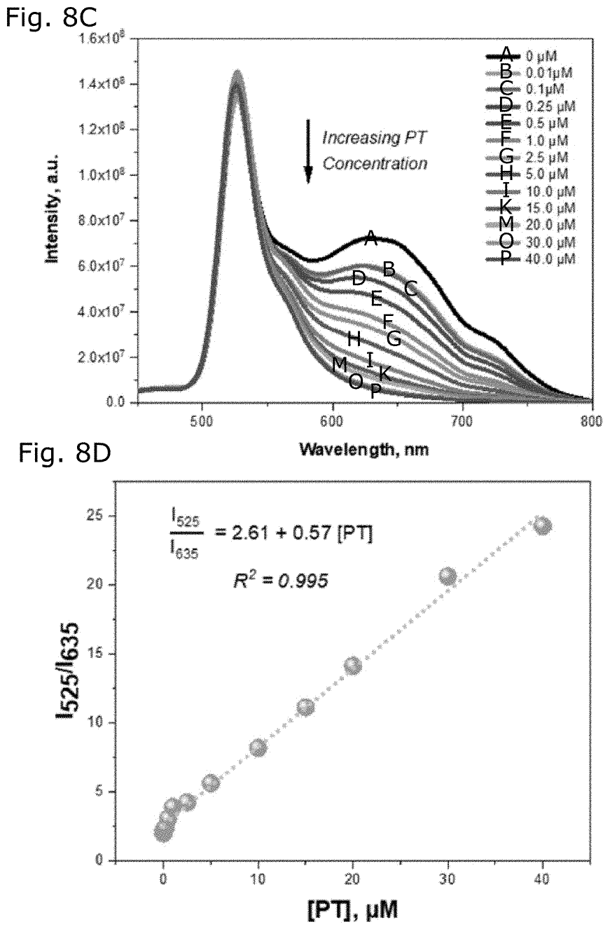

FIGS. 8A and 8C show the evolution of PL spectra of aqueous solutions of SiQDs and mAm mixtures (i.e., C_{SiQD}_{ₛ}=1.1 μM, C_{mAm}=1.8 μM) containing increasing concentrations of PX and PT, respectively. The SiQD PL intensity (λₘₐₓ=635 nm) is significantly diminished with increasing quencher concentration, whereas that of mAm (λₘₐₓ=525 nm) remains unchanged. A plot of the ratio of PL intensities at 525 and 635 nm (i.e., I₅₂₅/I₆₃₅) versus the concentrations of PX or PT yields linear relationships (FIGS. 8B and 8D) that point to the potential utility of SiQDs/mAm pairing in the analytical determination of PX and PT. To explore this possibility, we evaluated two solutions, one containing 25.0 μM PX and the other 25.0 μM PT; the presented calibration plots provided concentrations of 25.1 μM PX (0.26% error) and 25.1 μM PT (0.21% error), respectively. The limits of detection (LOD) of the present biocompatible metal-free SiQDs (LOD=3.3σ/slope, σ=standard deviation of the blank) for PX and PT obtained from the plots are 4.9 μM (1.3 μg mL−1) and 1.3 μM (0.38 pg mL−1), respectively, and are in the range of the minimum lethal dose (LD₅₀) of PT for human adults (20-100 mg) and mammals (e.g., mice, cats, dogs) (1-12 mg kg−1) and the reported amount of paraoxon that causes death of Sprague-Dawley rats in 6-8 min (4 mg kg−1).

The ratiometric sensing platform using mAm and red-photoluminescent SiQDs reported herein offers the advantage of operational simplicity as it does not depend on a cascade of chemical reactions catalyzed by enzymes for signal generation. Also, our detection strategy is straightforward and is, therefore, less likely to suffer from complications that might compromise sensor response (e.g., unwanted/unexpected loss of activity of enzymes due to denaturation or the presence of unknown inhibitors). Finally, the mAm-SiQD sensor allows for discrimination between nitrophenyl-based organophosphate ester nerve agents (e.g., parathion) and non-nitrophenyl-containing ones (e.g., diazinon). Our mAm-SiQD ratiometric detection system is simple and robust (i.e., a solution consisting of fluorescent/photoluminescent molecules and nanoparticles. Moreover, our sensor does not employ cytotoxic Cd-based QDs.

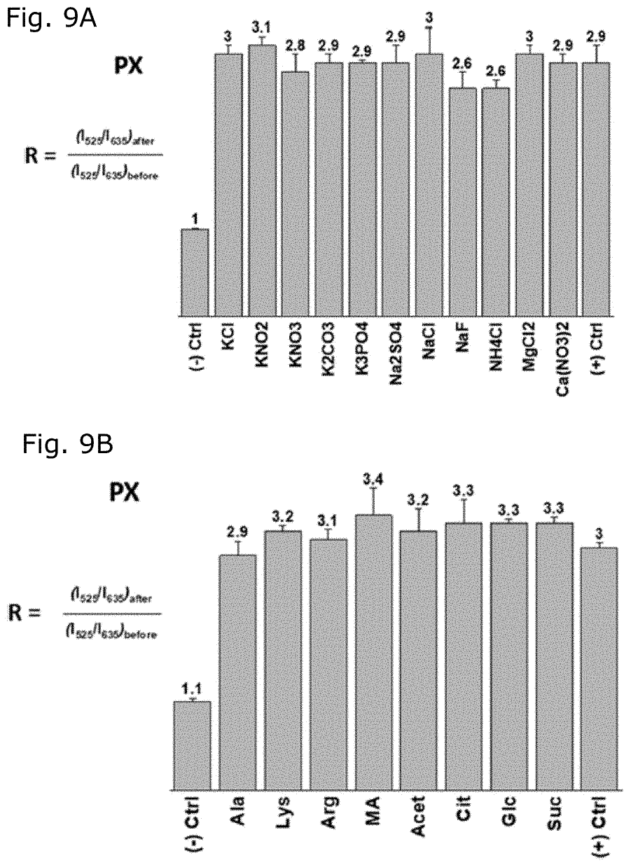

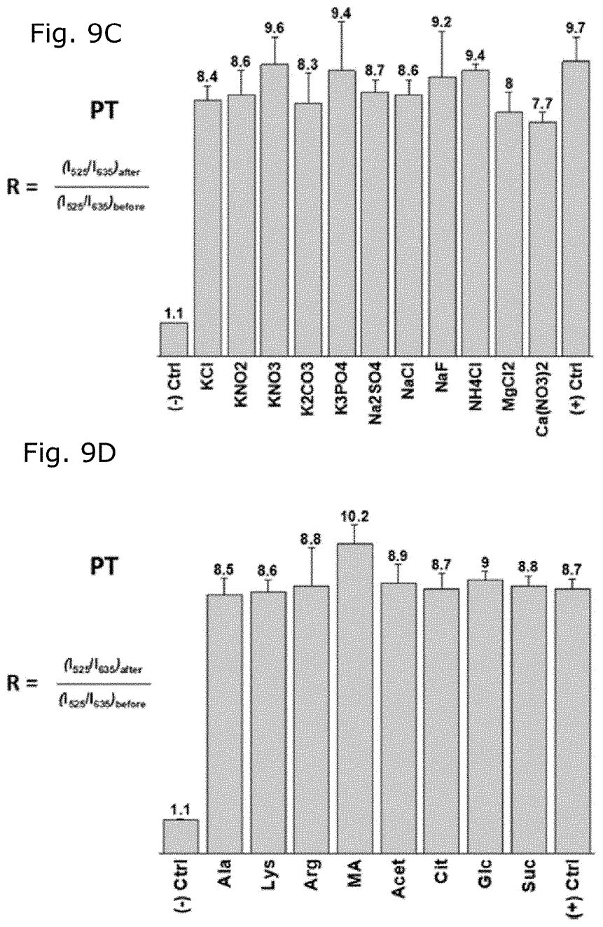

In an effort to evaluate the utility of the present sensing structure, we investigated the effects of different ions and organic species that are common interferents. This was achieved for the response to PX and PT by determining the ratios (R) of I₅₂₅/I₆₃₅ before and after the addition of the quenchers to an aqueous solution containing the interfering species (FIGS. 9A-D). All solutions yielded R values that differed significantly from the negative control (i.e., pure mQ water) and comparable to those of the corresponding positive controls (i.e., PX or PT in HEPES buffer). These observations indicate that PX and PT detection is relatively unaffected by the presence of inorganic and organic species. The values of R obtained for PT in the presence of divalent cations (i.e., Ca²⁺ and Mg²⁺) and malonic acid (MA) differ slightly from the (+) control. mAm, with a pKa of 5.8, is known to exhibit diminished fluorescence intensity under acidic conditions as a result of the protonation of its peptide-based fluorophore. This may explain why the R value of PT in the presence of MA differs slightly from that of the (+) control. The cause of the slight variations of PT R values in the presence of Ca²⁺ and Mg²⁺ ions compared to that of the (+) control is a subject of an ongoing investigation. These variations do not preclude the present sensing application.

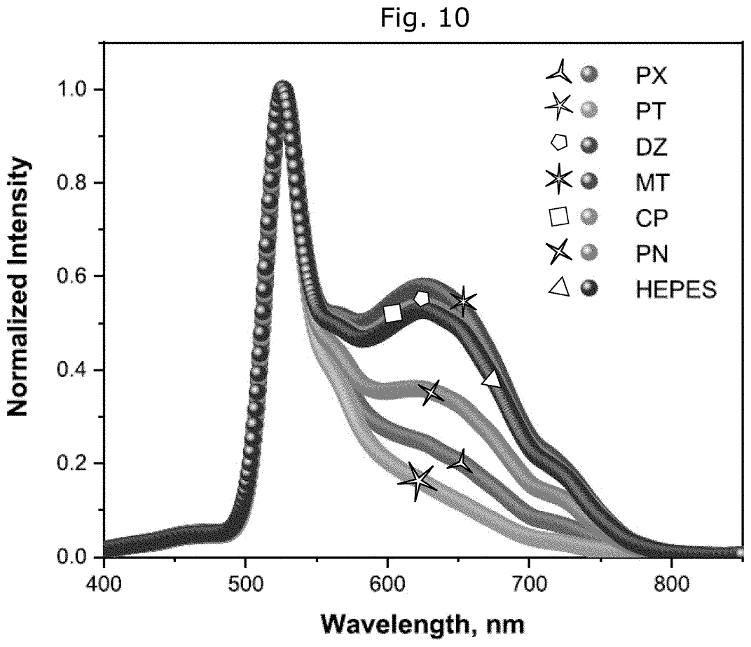

FIG. 10 shows that the ratiometric sensor is selective for PX, PT, and PN. The figure also shows that consistent with the Stern-Volmer plots, PT quenches the PL of the SiQDs best, followed by PX, and then by PN. The other organophosphates diazinon (DZ), malathion (MT), and chlorpyrifos (CP) did not quench the PL of the SiQDs presumably because they do not contain nitroaromatic groups.

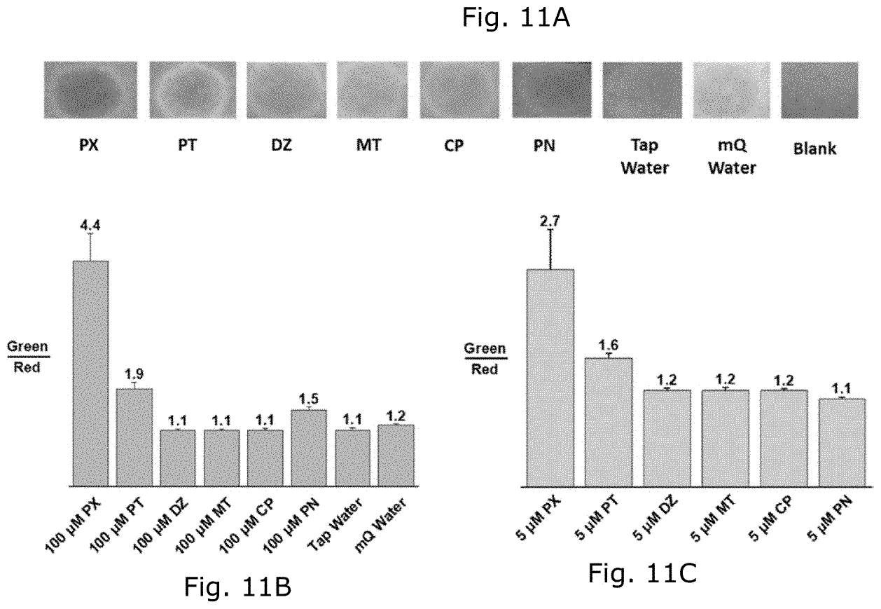

To expand the utility of the present sensing platform, we prepared paper-based sensors from SiQDs and mAm and used them to detect PX and PT. The papers were allowed to dry fully prior to use. FIG. 11A shows photographs of the paper-based sensors spotted with 100 μM of different organophosphates and PN, tap water, and mQ water under UV light illumination (λ=365 nm). The PL arising from spots exposed to PX, PT, and PN is qualitatively (visual inspection) more green than those of the other samples. To better quantify these findings, the emission from the spots was partitioned into red, green, and blue channels using a commercially available smartphone application (i.e., Color Picker™), and the ratio of green and red components was evaluated. The ratios obtained for PX and PT were 4.4 and 1.9, respectively, and are significantly larger compared to those obtained for mQ water (1.2) and tap water (1.1). We also note that the ratios obtained for spots arising from DZ, MT, and CP spots are near to that of mQ water, consistent with their inability to quench SiQD PL. Interestingly, PX appears to quench SiQD PL more strongly on paper than PT. This results from the relative lower polarity of PT which hinders it from effectively accessing the SiQDs that are supported in the hydrophilic cellulose network of the paper. FIG. 11C reveals that PX and PT can be detected and distinguished from water and other organophosphates even at concentrations as low as 5 μM. Also, PN can be detected at a concentration of 100 μM but not at 5 μM. These results support the implementation of the paper-based sensors developed herein as a quick and convenient litmus test for the detection of PX and PT.

Past attempts to produce bio-compatible quantum dots such as silicon quantum dots have encountered an issue of lack of sensitivity of silicon quantum dots; that is, they have been quenched by too many chemicals. The selectivity of the sensors tested within the class of organophosphates, and non-response to tap water, is considered an indication that the sensors will likely be selective to organophosphate esters containing nitroaromatic groups, or to compounds containing nitroaromatic groups in general, over a larger class of compounds.

### Experimental Conclusions

The present investigation reports the preparation of a convenient, biocompatible ratiometric photoluminescent sensor for paraoxon and parathion that is based on mAmetrine 1.2 and silicon quantum dots. PX and PT selectively quench SiQD photoluminescence by acting as dynamic quenchers. The ratiometric sensor developed has micromolar detection limits for PX and PT, is unaffected by inorganic and organic species, and is selective for PX and PT. Paper-based sensors containing mAm and SiQDs have also been used for detecting PX and PT at low concentrations. This sensor provides a straightforward and cost-effective system for direct detection of PX and PT by eliminating the need for intermediary biomolecules such as enzymes for signal generation, specificity, and selectivity.

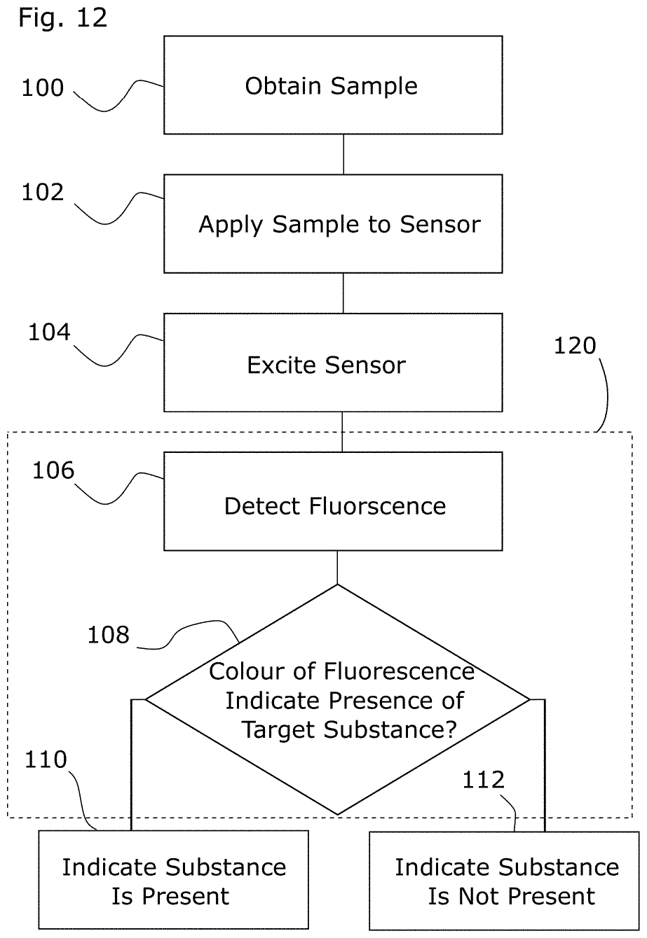

FIG. 12 illustrates a method of using a sensor according to the described embodiments, using automatic means to detect and analyze colour. The colour could also be detected by eye, and for example compared to a colour chart. In step 100 a sample is taken from a material. This sample may be a liquid sample or a solid, such as in the form of a swab or a piece of the material itself. In step 102 the sample is applied to the sensor. In the case of a liquid sample, this may be completed by applying one or more drops of the liquid sample to a sensor surface on a paper substrate. In an example embodiment, a sensor may be provided on a substrate as a pair of surfaces, the first being a reference surface and the second being the sensing surface. In one such embodiment, the two surfaces are prepared as approximately adjacent regions on a strip of sampling paper. A liquid sample could also be introduced to sensor in liquid solution. A solid sample, such as a swab may similarly be applied to a sensor solution.

In step 104 the fluorescent materials of the sensor are excited by the application of suitable electromagnetic radiation. In the case of the materials described in the experimental set up, ultraviolet from an ultraviolet light source may be used to excite the sensor to emit fluorescence.

In step 106 the fluorescence from the sample is detected. Various types of light detector, e.g. cameras, may be suitable for detecting the fluorescence of the sensor. In some embodiments, a smartphone 120 camera may be used.

In step 108 the fluorescence detected is analyzed. The detected fluorescence may be compared against a reference sensor, i.e. a sensor without an applied sample. As described previously, the sensor materials are sensitive to the presence of one or more substances which, if present, will quench the fluorescence of the biologically-compatible quantum dots. The quenching of the fluorescence produces a colour difference between the light produced by the sensor with the sample versus the reference sensor. In some embodiments, instead of a reference sensor there may be a reference baseline in internal of the analyzing device. The analysis of the fluorescence may be performed by a suitable processor connected to receive the detected fluorescence from the light sensor. In some embodiments, a smartphone 120 colour analysis app may be used to analyze the fluorescence.

In steps 110 and 112 an output is produced concluding whether the substances are present based on the analysis of the fluorescence. For example, in a sensor constructed according to the experimental setup described above, this may include a conclusion as to whether an organophosphate ester is present.

According to an embodiment, a sensor may be produced by preparing a combination of biologically compatible quantum dots with an organic fluorophore. Preparation of biologically compatible quantum dots may comprise, for example, 10-undecenoic acid and allyloxy poly(ethylene oxide) methyl ether linked to the surfaces of 6 nm hydride terminated silicon nanoparticles via thermally induced hydrosilylation. Organic fluorophores may be produced any of a variety of processes. In one process, DNA encoding mAmetrine 1.2 in pBAD/His B vector is transformed into electrocompetent Escherichia coli strain DH10B. Bacteria may be cultured, harvested, lysed, and clarified. The cleared lysate is then incubated, washed and eluted. Purified mAmetrine 1.2 may be extracted from the solution. A combination of biologically compatible quantum dots with an organic fluorophore may be applied in a sensor either as a solution or as a sensing surface on a solid substrate.

The following documents are incorporated by reference:

Robidillo, C. J. T.; Wandelt, S.,; Dalangin, R.; Zhang, L.; Yu, H. Y.; Meldrum, A.; Campbell, R. E.; Veinot, J. G. C. Ratiometric Detection of Nerve Agents by Coupling Complementary Properties of Silicon-Based Quantum Dots and Green Fluorescent Protein. ACS Appl. Mater. Interfaces 2019, 11, 33478-33488.

Meng, X.; Wei, J.; Ren, X.; Ren, J.; Tang, F. A Simple and Sensitive Fluorescence Biosensor for Detection of Organophosphorus Pesticides Using H2O2-Sensitive Quantum Dots/Bi-Enzyme. Biosens. Bioelectron. 2013, 47, 402-407.

Zheng, Z.; Zhou, Y.; Li, X.; Liu, S.; Tang, Z. Highly-Sensitive Organophosphorous Pesticide Biosensors Based on Nanostructured Films of Acetylcholinesterase and CdTe Quantum Dots. Biosens. Bioelectron. 2011, 26, 3081-3085.

Yan, X.; Li, H.; Yan, Y.; Su, X. Selective Detection of Parathion- Methyl Based on near-Infrared CuInS2 Quantum Dots. Food Chem. 2015, 173, 179-184.

McVey, B. F.; Tilley, R. D. Solution Synthesis, Optical Properties, and Bioimaging Applications of Silicon Nanocrystals. Acc. Chem. Res. 2014, 47, 3045-3051.

Zhai, Y.; Dasog, M.; Snitynsky, R. B.; Purkait, T. K.; Aghajamali, M.; Hahn, A. H.; Sturdy, C. B.; Lowary, T. L.; Veinot, J. G. C. Water-Soluble Photoluminescent D-Mannose and L-Alanine Functionalized Silicon Nanocrystals and Their Application to Cancer Cell Imaging. J. Mater. Chem. B 2014, 2, 8427-8433.

Gonzalez, C. M.; Iqbal, M.; Dasog, M.; Piercey, D. G. Lockwood, R.; Klapotke, T. M.; Veinot, J. G. C. Detection of High-ACS Applied Materials & Interfaces Research Article DOI: 10.1021/acsami.9b10996 ACS Appl. Mater. Interfaces 2019, 11, 33478-33488 33487 Energy Compounds Using Photoluminescent Silicon Nanocrystal Paper Based Sensors. Nanoscale 2014, 6, 2608-2612.

Sinelnikov, R.; Dasog, M.; Beamish, J.; Meldrum, A.; Veinot, J. G. C. Revisiting an Ongoing Debate: What Role Do Surface Groups Play in Silicon Nanocrystal Photoluminescence? ACS Photonics 2017, 4, 1920-1929.

Islam, M. A.; Sinelnikov, R.; Howlader, M. A.; Faramus, A.; Veinot, J. G. C. Mixed Surface Chemistry: An Approach to Highly Luminescent Biocompatible Amphiphilic Silicon Nanocrystals. Chem. Mater. 2018, 30, 8925-8931.

Liu, J.; Erogbogbo, F.; Yong, K.-T.; Ye, L.; Liu, J.; Hu, R.; Chen, H.; Hu, Y.; Yang, Y.; Yang, J.; et al. Assessing Clinical Prospects of Silicon Quantum Dots: Studies in Mice and Monkeys. e 2013, 7, 7303-7310.

Chen, N.; He, Y.; Su, Y.; Li, X.; Huang, Q.; Wang, H.; Zhang, X.; Tai, R.; Fan, C. The Cytotoxicity of Cadmium-Based Quantum Dots. Biomaterials 2012, 33, 1238-1244.

Cubitt, A. B.; Heim, R.; Adams, S. R.; Boyd, A. E.; Gross, L. A.; Tsien, R. Y. Understanding, Improving and Using Green Fluorescent Proteins. Trends Biochem. Sci. 1995, 20, 448-455.

Ormö, M.; Cubitt, A. B.; Kallio, K.; Gross, L. A.; Tsien, R. Y.; Remington, S. J. Crystal Structure of the Aequorea Victoria Green Fluorescent Protein. Science 1996, 273, 1392-1395.

Miyawaki, A.; Llopis, J.; Heim, R.; McCaffery, J. M.; Adams, J. A.; Ikura, M.; Tsien, R. Y. Fluorescent Indicators for Ca2+ Based on Green Fluorescent Proteins and Calmodulin. Nature 1997, 388, 882-887.

Miyawaki, A.; Niino, Y. Molecular Spies for Bioimaging—Fluorescent Protein-Based Probes. Mol. Cell 2015, 58, 632-643.

Ding, Y.; Ai, H.; Hoi, H.; Campbell, R. E. Föster Resonance Energy Transfer-Based Biosensors for Multiparameter Ratiometric Imaging of Ca2+ Dynamics and Caspase-3 Activity in Single Cells. Anal. Chem. 2011, 83, 9687-9693.

Hessel, C. M.; Henderson, E. J.; Veinot, J. G. C. Hydrogen Silsesquioxane: A Molecular Precursor for Nanocrystalline Si—SiO2 Composites and Freestanding Hydride-Surface-Terminated Silicon Nanoparticles. Chem. Mater. 2006, 18, 6139-6146.

Robidillo, C. J. T.; Aghajamali, M.; Faramus, A.; Sinelnikov, R.; Veinot, J. G. C. Interfacing Enzymes with Silicon Nanocrystals through the Thiol-Ene Reaction. Nanoscale 2018, 10, 18706-18719.

Robidillo, C. J. T.; Islam, M. A.; Aghajamali, M.; Faramus, A.; Sinelnikov, R.; Zhang, X.; Boekhoven, J.; Veinot, J. G. C. Functional Bioinorganic Hybrids from Enzymes and Luminescent Silicon-Based Nanoparticles. Langmuir 2018, 34, 6556-6569.

Otsuka, H.; Nagasaki, Y.; Kataoka, K. PEGylated Nanoparticles for Biological and Pharmaceutical Applications. Adv. Drug Delivery Rev. 2012, 64, 246-255.

Pelaz, B.; del Pino, P.; Maffre, P.; Hartmann, R.; Gallego, M.; Rivera-Fernandez, S.; de la Fuente, J. M.; Nienhaus, G. U.; Parak, W. J. Surface Functionalization of Nanoparticles with Polyethylene Glycol: Effects on Protein Adsorption and Cellular Uptake. ACS Nano 2015, 9, 6996-7008.

Dai, Q.; Walkey, C.; Chan, W. C. Polyethylene Glycol Backfilling Mitigates the Negative Impact of the Protein Corona on Nanoparticle Cell Targeting. Angew. Chem., Int. Ed. 2014, 53, 5093-5096.

Wörz, A.; Berchtold, B.; Moosmann, K.; Prucker, O.; Rühe, J. Protein-Resistant Polymer Surfaces. J. Mater. Chem. 2012, 22, 19547-19561.

Clark, R. J.; Dang, M. K.; Veinot, J. G. C. Exploration of Organic Acid Chain Length on Water-Soluble Silicon Quantum Dot Surfaces. Langmuir 2010, 26, 15657-15664.

Mkhatresh, O. A.; Heatley, F. A Study of the Products and Mechanism of the Thermal Oxidative Degradation of Poly (Ethylene Oxide) Using 1H and 13C 1-D and 2-D NMR. Polym. Int. 2004, 53, 1336-1342.

Yang, L.; Heatley, F.; Blease, T. G.; Thompson, R. I. A Study of the Mechanism of the Oxidative Thermal Degradation of Poly (Ethylene Oxide) and Poly (Propylene Oxide) Using 1H-and 13CNMR. Eur. Polym. J. 1996, 32, 535-547.

Matsuura, H.; Miyazawa, T. Vibrational Analysis of Molten Poly (Ethylene Glycol). J. Polym. Sci., Part A-2 1969, 7, 1735-1744.

Yang, Z.; Gonzalez, C. M.; Purkait, T. K.; Iqbal, M.; Meldrum, A.; Veinot, J. G. C. Radical Initiated Hydrosilylation on Silicon Nanocrystal Surfaces: An Evaluation of Functional Group Tolerance and Mechanistic Study. Langmuir 2015, 31, 10540-10548.

Doremus, R. H. Oxidation of Silicon by Water and Oxygen and Diffusion in Fused Silica. J. Phys. Chem. A. 1976, 80, 1773-1775.

Flitsch, R.; Raider, S. I. Electron Mean Escape Depths from Xray Photoelectron Spectra of Thermally Oxidized Silicon Dioxide Films on Silicon. J. Vac. Sci. Technol. 1975, 12, 305-308.

Damodaran, V. B.; Fee, C. J.; Ruckh, T.; Popat, K. C. Conformational Studies of Covalently Grafted Poly (Ethylene Glycol) on Modified Solid Matrices Using X-Ray Photoelectron Spectroscopy. Langmuir 2010, 26, 7299-7306.

Briggs, D.; Beamson, G. Primary and Secondary Oxygen-Induced C1s Binding Energy Shifts in x-Ray Photoelectron Spectroscopy of Polymers. Anal. Chem. 1992, 64, 1729-1736.

Thomas, H. R.; O'Malley, J. J. Surface Studies on Multicomponent Polymer Systems by X-Ray Photoelectron Spectroscopy. Polystyrene/Poly (Ethylene Oxide) Diblock Copolymers. Macromolecules 1979, 12, 323-329.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

1. A sensor for detecting a substance, the sensor comprising:
a combination of biocompatible quantum dots and an organic fluorophore in a controlled ratio, the organic fluorophore exhibiting fluorescence of a first color, the biocompatible quantum dots sized to exhibit fluorescence of a second color different from the first color, and the biocompatible quantum dots functionalized with an organic coating arranged to chemically interact with the substance to quench the fluorescence of the biocompatible quantum dots.

2. The sensor of claim 1 in which the biocompatible quantum dots are silicon nanoparticle quantum dots.

3. The sensor of claim 1 in which the organic fluorophore is a green fluorescent protein.

4. The sensor of claim 3 in which the green fluorescent protein is mAmetrine 1.2.

5. The sensor of claim 1 in which the substance contains a nitroaromatic group.

6. The sensor of claim 5 in which the substance is an organophosphate ester containing a nitroaromatic group.

7. The sensor of claim 1 in which the organic coating comprises poly(ethylene oxide).

8. The sensor of claim 7 in which the poly(ethylene oxide) terminates in an alkoxide group where the pendant alkyl group is a C1, C2, C3, C4, C5, C6, C7, C8, C9, C10, C11, or C12 alkyl group.

9. The sensor of claim 8 in which the poly(ethylene oxide) terminates in a methyl ether group.

10. The sensor of claim 1 in which the organic coating comprises a water solubility enhancing component.

11. The sensor of claim 10 in which the water solubility enhancing component comprises one or more of a carboxylic acid, a primary amine, a secondary amine, or an alcohol.

12. The sensor of claim 11 in which the organic coating comprises a carboxylic acid.

13. The sensor of claim 12 in which the organic coating comprises carboxylic acids including more than one type of alkyl group.

14. The sensor of claim 12 in which the organic coating comprises carboxylic acid with a C12 alkyl group.

15. The sensor of claim 12 in which the organic coating comprises 10-undecenoic acid.

16. The sensor of claim 1 in which the substance comprises one or more of paraoxon, parathion, or p-nitrophenol.

17. The sensor of claim 1 including the combination in a solution.

18. The sensor of claim 1 including the combination on a solid carrier.

19. The sensor of claim 18 in which the solid carrier comprises one of a paper, a polymer fiber membrane, a glass fiber membrane, or a polymer substrate.

20. The sensor of claim 1 comprising:
a light sensor situated to detect fluorescence of the combination of the biocompatible quantum dots and the organic fluorophore; and
a processor connected to the light sensor to analyze the detected fluorescence.

21. A method of sensing a substance comprising:
providing a combination of biologically-compatible quantum dots and an organic fluorophore in a controlled ratio, the organic fluorophore exhibiting fluorescence of a first color, the biologically-compatible quantum dots sized to exhibit fluorescence of a second color different from the first color, and the biologically-compatible quantum dots functionalized with an organic coating arranged to chemically interact with the substance to quench the fluorescence of the biologically-compatible quantum dots;
applying a sample of a material to the combination of the biologically-compatible quantum dots and the organic fluorophore;
exciting the combination of the biologically-compatible quantum dots and the organic fluorophore;
detecting a produced fluorescence; and
determining whether the produced fluorescence indicates a presence of the substance.

22. A sensor for detecting a substance, the sensor comprising:
a combination of a biocompatible fluorescent nanoparticle that is responsive to the substance and an organic fluorophore in a controlled ratio, the organic fluorophore stable with respect to the substance, the organic fluorophore exhibiting fluorescence of a first color, the biocompatible fluorescent nanoparticle exhibiting fluorescence of a second color different from the first color, and the biocompatible fluorescent nanoparticle functionalized with an organic coating arranged to chemically interact with the substance to quench the fluorescence of the biocompatible fluorescent nanoparticle.
